(19) United States
(12) Patent Application Publication
(10) Pub. No.: US 2003/0091978 A1
(43) Pub. Date: May 15, 2003

(54) Assay for glucocorticoid receptor signalling pathway

(76) Inventors: Stuart Neville Farrow, Stevenage (GB); Barbara Maschera, Stevenage (GB); Edward Stephen Rees, Stevenage (GB)

Correspondence Address: DAVID J LEVY, CORPORATE INTELLECTUAL PROPERTY, GLAXOSMITHKLINE, FIVE MOORE DR., PO BOX 13398, RESEARCH TRIANGLE PARK, NC, 27709-3398, US

(21) Appl. No.: 10/182,015

(22) PCT Filed: Jan. 24, 2001
(86) PCT No.: PCT/GB01/00286

(30) Foreign Application Priority Data
Jan. 25, 2000 (GB) 0001703.8

(51) Int. Cl. C12Q 1/00; C12Q 1/68

(52) U.S. Cl. 435/4; 435/6

(57) ABSTRACT

The present invention provides a method of assessing the glucocorticoid receptor signalling pathway modulated by a test compound comprising; (a) providing a cell having a first polynucleotide construct comprising a first reporter gene under the control of a first promoter indicative of a first glucocorticoid receptor signalling pathway; (b) providing a second cell of the same lineage as the cell (a), and comprising a second polynucleotide construct having a second reporter gene under the control of a second promoter indicative of a second glucocorticoid receptor signalling pathway; (c) incubating said first and second cells with a test compound; (d) monitoring for expression of the first and second reporter genes.

### FIELD OF THE INVENTION

The invention relates to an assay for assessing the signal pathway modulated in response to a compound acting at a nuclear receptor and in particular a glucocorticoid receptor.

### BACKGROUND OF THE INVENTION

Glucocorticoids are useful therapeutic agents. In particular, glucocorticoids are potent anti-inflammatory drugs. However, administration of glucocorticoids or analogues thereof generally leads to unwanted side-effects which limit their therapeutic use.

Binding of glucocorticoids to a receptor may lead to modulation of cellular events through more than one signal transduction pathway. For example, glucocorticoids bind to a cytosolic nuclear receptor (termed GR), leading to its nuclear translocation followed by modulation of gene transcription. Transcriptional activation and repression may occur either by direct DNA binding or by an indirect inhibition of key transcription factors such as AP-1 and NF-κB.

It is thought that the beneficial anti-inflammatory effects of glucocorticoids are mediated by transcriptional repression without direct DNA binding of the GR. Thus, steroid analogues that distinguish between the two activation pathways may have an improved therapeutic profile. In particular analogues that selectively modulate a single signal transduction pathway but do not have any effect on the other pathway will be useful therapeutic agents.

Some glucocorticoid analogues have been identified that exert strong AP-1 inhibition but show little or no transactivation through direct DNA binding. There is a need to identify such agents in an assay which is able to distinguish between the two signal pathways.

### SUMMARY OF THE INVENTION

The present inventors have now developed a screening method or assay which can be used to assess the nuclear receptor signalling pathway modulated by a test compound.

In a first aspect, the invention provides a method of assessing glucocorticoid receptor signalling pathway modulated by a test compound comprising;

(a) providing a cell having a first polynucleotide construct comprising a first reporter gene under the control of a first promoter indicative of a first glucocorticoid receptor signalling pathway;

(b) providing a second cell of the same lineage as the cell (a), and comprising a second polynucleotide construct having a second reporter gene under the control of a second promoter indicative of a second glucocorticoid signalling pathway;

(c) incubating said first and second cells with the test compound; and

(d) monitoring for expression of the first and second reporter genes.

In preferred aspects of the invention, the first and/or second promoters comprise a responsive element indicative of the signalling pathway under investigation. The first or second promoter may comprise a glucocorticoid responsive element which is activated by direct DNA binding or a transcription factor binding site such as an AP-1 recognition site or an NF-κB recognition site. The promoter may comprise a non-naturally occurring promoter comprising a minimal promoter together with the responsive element.

In one aspect of the invention, the first and second reporter genes are selected so that they are detectable by fluorogenic, luminometric or colourometric means. Preferably, both reporter genes are detectable by luminometric means and may preferably comprise firefly-luciferase and renilla-luciferase.

The first and second polynucleotide constructs are provided in cells of the same lineage. They may be provided in the same cell.

### FIGURES

FIG. 1 shows dose-dependent inhibition of NF-κB driven firefly-luciferase activity by dexamethasone in A549-NF-κB-firefly cells.

FIG. 2 shows dose-dependent inhibition of AP-1 driven firefly-luciferase activity by dexamethasone in A549-API-firefly cells.

FIG. 3 shows dose-dependent increase in renilla luciferase activity as a result of stimulation with dexamethasone.

FIG. 4 shows dose-dependent increase in renilla luciferase activity as a result of stimulation with dexamethasone.

FIG. 5 shows dose dependent inhibition of renilla luciferase activity and increase in firefly luciferase activity as a result of stimulation with dexamethasone.

### DETAILED DESCRIPTION OF THE INVENTION

The invention relates to a cell-based assay in which first and second cells are provided which have first and second polynucleotide constructs. Each construct comprises a reporter gene under the control of a promoter which is responsive to a signalling pathway.

The constructs may comprise DNA or RNA. They may also include within them synthetic or modified nucleotides. A number of different types of modification to polynucleotides are known in the art. These include methylphosphonate and phosphorothioate backbones, addition of acridine or polylysine chains at the 3′ and/or 5′ ends of the molecule. For the purposes of the invention, it is to be understood that the constructs described herein may be modified by any method available in the art. Such modifications may be carried out in order to enhance the in vivo activity or stability of constructs of the invention. Constructs of the invention may be produced recombinantly, synthetically, or by any means available to those of skill in the art.

The term “operably linked” refers to a juxtaposition wherein the components described are in a relationship permitting them to function in their intended manner. Thus, a regulatory sequence, such as a promoter, “operably linked” to a coding sequence is positioned in such a way that expression of the coding sequence is achieved under conditions compatible with the regulatory sequence.

A promoter for use in a construct of the invention is a promoter which can drive the transcription of the reporter gene to which it is operably linked. The first and second promoters are responsive to modulation of a signalling pathway. The first and second promoters are responsive to different signalling pathways.

Examples of signalling pathways which might be monitored in accordance with the invention are glucocorticoid receptor signalling via transcription factors such as AP-1 and NF-κB, C/EBP-1, STAT-5, Oct-1 and Oct-2. Alternatively, the signalling pathway may comprise direct DNA binding, via a glucocorticoid responsive element (GRE).

The assay of the invention may be used to compare at least two signalling pathways. The assay may be used to compare signalling via, for example, GRE and NF-κB, GRE and AP-1, GRE and C/EBP-1, GRE and STAT-5, GRE and Oct-1 and GRE and Oct-2.

The assay of the invention may be used to compare signalling via more than two pathways and thus may be used to compare signalling via 3 or more, for example 3, 4 or 5 pathways. The assay may be used to compare signalling via, for example, NF-κB, GRE and AP-1.

The promoter comprises at least one region which is responsive to activation by the pathway to be monitored. For example, the promoter may comprise part or all of the glucocorticoid responsive element, an AP-1 recognition site or an NF-κB recognition site or recognition sites for C/EBP-1, STAT-5, Oct-1 or Oct-2 or other transcription factor recognition sites involved in glucocorticoid receptor signalling or a complete promoter. The promoter may comprise a non-naturally occurring promoter comprising a minimal promoter together with a signalling-pathway responsive element.

The first promoter linked to a first reporter gene and the second promoter linked to the second reporter gene thus are indicative of alternative pathways which may be mediated by a glucocorticoid acting at a glucocorticoid nuclear receptor. The promoter may comprise a promoter from an endogenous gene in which transcription factors such as AP-1 and NF-κB, C/EBP-1, STAT-5, Oct-1 or Oct-2 are involved in transcription of the gene from the promoter. Alternatively, the promoter may comprise the endogenous glucocorticoid receptor (GR) normal promoter. In these constructs, the entire promoter from the endogenous gene will be incorporated into the polynucleotide construct for expression of the reporter gene.

In an alternative embodiment, the promoter may comprise a non-naturally occurring promoter comprising part or all of an endogenous promoter with or without additional promoter sequences. For example, some endogenous promoters may incorporate more than one NF-κB or AP-1, C/EBP-1, STAT-5, Oct-1 or Oct-2 or other transcription factor recognition sites. The promoters may also include non-essential sequences which are not necessarily required for expression of the gene. Thus, a promoter sequence which includes a deletion, such as a deletion of one of the transcription factor such as AP-1 or NF-κB recognition sites, or of non-essential sequences may be used in accordance with the invention so long as the promoter does include at least one transcription factor recognition sites. Similarly, the promoter may comprise an endogenous promoter into which additional transcription factor recognition sites, such as AP-1 or NF-κB, C/EBP-1, STAT-5, Oct-1 or Oct-2 recognition sites or GRE recognition sites have been introduced. Such promoters may be useful to enhance the signal that is generated from a reporter gene. A promoter may comprise a non-naturally occurring promoter comprising a minimal promoter together with a signalling pathway responsive element. Suitable minimal promoters include a minimal herpes simplex thymidine kinase promoter. Additional enhancer sequences may be incorporated. More than one recognition site such as several AP-1 recognition sites may be incorporated into the minimal promoter, again to enhance the signal that is produced.

The promoter is operably linked to a reporter gene which encodes a protein which can be readily detected by standard methods. For example, the reporter product may be detectable by fluorescent, luminescent or other standard reporting techniques. The reporter gene may comprise an enzyme such as β-galactosidase, production of which may be identified by use of a colourogenic or fluorogenic enzyme substrate. Other reporter genes include β-glucuronidase, green fluorescent protein (GFP) and variants thereof, luciferase, chloramphenicol acetyl transferase, catechol oxidase, an antigen which may readily be recognised by an antibody, other affinity ligands such as streptavidin/biotin or protein A which may be detected by antibodies etc. The first and second reporter genes are selected such that it is possible to differentiate between expression of the first reporter gene and expression of the second reporter gene. In a preferred aspect of the invention, both of the reporter genes comprise a luciferase. A first luciferase is the firefly-luciferase and the second luciferase comprises the renilla-luciferase. Firefly and renilla luciferase can readily be distinguished from each other in a luminescent assay.

Constructs of the invention can be incorporated into a recombinant vector. For example, a recombinant replicable vector may be provided to replicate the construct in a compatible host cell. The vector may be, for example, plasmid, virus or phage vector. The vectors may contain one or more selectable marker genes such as a neomycin resistance gene for selection in mammalian cells.

The vectors of the invention which incorporate a reporter gene under the control of a promoter responsive to a signalling pathway may be introduced into a suitable host cell by any appropriate transformation or transfection techniques. Preferably, the host cell will permit expression of the reporter gene and will also contain the appropriate machinery for the signalling pathway under investigation. In particular therefore the cells are mammalian cells. It is important that the first and second reporter genes are provided in cells of the same cell lineage. This removes any differential expression which may be seen using cells from different cell lines or different responsiveness to a compound under test through differential expression of components of the signalling pathway under investigation.

In addition, some cell types show differential uptake of compounds under assay. For example yeast cells are resistant to uptake of glucocorticoids. Differences in the uptake of the compound under test by cells of different lineage may lead to distortion of the results seen in the assay which do not reflect differential signalling. Similarly, the same compound may show different stability in different cells. In some instances, it may be possible to use cells of a different lineage in accordance with the assay. However, for such cells, a compound under test should be assayed first using the same reporter system under the expression of the same promoter in each of the two cell types to confirm that there is no differential stability, expression or uptake of the compound under test associated with the different cell types.

A cell for use in accordance with the assay expresses the glucocorticoid receptor. A cell for use in the invention may comprise a cell which does not usually express the glucocorticoid receptor but which has been transformed or transfected with a polynucleotide construct encoding the glucocorticoid receptor preferably under the control of a constitutive promoter to ensure that the receptor is expressed in those cells. Alternatively, an inducible promoter may be used, which is induced prior to or during the assay.

Preferred cells for use in accordance with the invention includes epithelial cells, embryonic kidney cells or liver specific cells such as A549 or A375 cells, HEK293 cells or H4IIEC3 cells, eosinophils and neutrophils, primary human T-cells and primary synoviocytes. In a preferred aspect of the invention, the cell line to be used in the assay is selected depending on the proposed use of the compound. For example, if the compound is proposed for use in the treatment of inflammation of a mucosal surface, epithelial cells could be used. Compounds proposed for use in the treatment of asthma may be assayed using eosinophils or neutrophils for example or compounds proposed for use in the treatment of rheumatoid arthritis may be assayed in primary human T-cells or primary synoviocytes. In an alternative aspect the first and second reporter genes are provided in a single cell.

An assay of the invention is used to asses which signalling pathway is modulated by a compound under test acting at a glucocorticoid receptor. Preferably, a compound under test is an analogue of a glucocorticoid. The compound under test may be a known glucocorticoid analogue or may be a new glucocorticoid analogue. The assay is thus used to monitor or identify the signalling pathway which is mediated by the compound under test/glucocorticoid receptor interaction. Glucocorticoid analogues may comprise a steroid or may be a non-steroid compound which may nevertheless bind or interact with the glucocorticoid receptor.

In an assay of the invention, a first cell and a second cell are provided having therein a polynucleotide construct having a reporter gene under the control of a promoter responsive to a signalling pathway.

Any suitable assay format may be used. The assay is, however, typically carried out in a single medium to minimise or remove differences in culture conditions etc by carrying out the assay in separate mediums. Most preferably, the assay is carried out in a single well of a microtiterplate, so that high throughput screening may be carried out. Typically, the first and second cells are provided and are incubated with a compound under test over a desired period of time. Glucocorticoids modulate gene transcription by indirect inhibition of the transcription factors such as AP-1 or NF-κB. Thus, in order to establish whether such inhibition is occurring with a compound under test, it is generally necessary to stimulate the relevant transcription factor and thus determine whether a compound under test inhibits such activated transcription. This may be done by incubating the cells with a stimulatory factor such as IL-1, TNF, EGF or other factors which activate transcription factors such as NF-κB or AP-1, C/EBP-1, STAT-5, Oct-1 or Oct-2. Preferably step (c) of the assay comprises incubating the first, second and any additional cells with a stimulatory factor and then incubating the first, second and any additional cells with a test compound.

The assay may be carried out in the presence of agents which mediate transcription through both the first and second signalling pathways to demonstrate the expression pattern that may be seen for the first and second reporter genes. If available, test substances which only activate through a single transcription pathway may also be used as a control in order to assess differential activity for comparison with a compound under test.

Typically, a compound under test will be incubated with the cells of the invention over a period of from 1 to 24 hours. Subsequently, transcription mediated by factors such as AP-1 or NF-κB is activated by the addition of TNF or other suitable agent over a period of from 8 to 24 hours and expression of the reporter genes is then assayed. The assay may also be carried out in the absence of any substance under test to monitor for successful activation of transcription via the transcription factor.

Suitable substances which can be tested in the above assays are analogues of compounds known to act at a receptor under investigation. Compounds under test may be steroids such as glucocorticoids or non steroidal compounds.

Test substances may be used at a concentration of from 1 nM to a 1000:M, preferably from 1 nM to 100:M, more preferably from 1 nM to 10:M. A substance will preferably show differential expression of the first and second reporter genes. Preferably, a compound under test will show at least 2 fold activity, more preferably 5 fold, 10 fold, 20 fold or 100 fold, 200 fold or 500 fold or more expression of a first reporter gene relative to expression of the second reporter gene.

Compounds demonstrating differential activation of the glucocorticoid receptor may be used in the treatment of a variety of conditions. In particular, the invention provides specific or selective compounds for which the first signalling pathway is associated with a beneficial therapeutic effect and the second signalling pathway, which is not activated by the compound, is associated with detrimental or unwanted side effects. Preferably, the invention is used to identify glucocorticoid and non-steroidal compounds that modulate transcription by indirect inhibition of a transcription factor, such as AP-1 or NF-6B but which do not show transcriptional activation by direct DNA binding at the GR recognition site. Such compounds may be used in the treatment of rheumatoid arthritis, osteoarthritis, inflammatory pain, emesis, inflammatory bowel disease, asthma, atopic dermatitis and psoriasis.

Compounds identified in accordance with the invention may be administered in a variety of dosage forms. Thus, they can be administered orally, for example as tablets, lozenges, aqueous or oily suspensions, dispersible powders or granules. Such modulators may also be administered parenterally, either subcutaneously, intravenously, intramuscularly, transdermally or by infusion techniques, or topically in particular to the skin. A physician may be able to determine the required route of administration for each particular patient. The formulation of a compound for use in prophylaxis or treatment would depend on factors such as the nature of the substance and the intended use.

A desired substance is typically formulated for administration in the present invention with a pharmaceutically acceptable carrier or diluent. The pharmaceutical carrier or diluent may be, for example, an isotonic solution. For example, solid oral forms may contain, together with the active compound, diluents, e.g. lactose, dextrose, saccharose, cellulose, corn starch or potato starch; lubricants, e.g. silica, talc, stearic acid, magnesium or calcium stearate, and/or polyethylene glycols; binding agents; e.g. starches, arabic gums, gelatin, methylcellulose, carboxymethylcellulose or polyvinyl pyrrolidone; disaggregating agents, e.g. starch, alginic acid, alginates or sodium starch glycolate; effervescing mixtures; dyestuffs; sweeteners; wetting agents, such as lecithin, polysorbates, laurylsulphates; and, in general, non-toxic and pharmacologically inactive substances used in pharmaceutical formulations. Such pharmaceutical preparations may be manufactured in known manner, for example, by means of mixing, granulating, tabletting, sugar-coating, or film coating processes.

Liquid dispersions for oral administration may be syrups, emulsions and suspensions. The syrups may contain as carriers, for example, saccharose or saccharose with glycerine and/or mannitol and/or sorbitol.

Suspensions and emulsions may contain as carrier, for example a natural gum, agar, sodium alginate, pectin, methylcellulose, carboxymethylcellulose, or polyvinyl alcohol. The suspensions or solutions for intramuscular injections may contain, together with the active compound, a pharmaceutically acceptable carrier, e.g. sterile water, olive oil, ethyl oleate, glycols, e.g. propylene glycol, and if desired, a suitable amount of lidocaine hydrochloride.

Solutions for intravenous or infusions may contain as carrier, for example, sterile water or preferably they may be in the form of sterile, aqueous, isotonic saline solutions.

A therapeutically effective amount of a modulator is administered to a patient. The dose of modulator may be determined according to various parameters, especially according to the substance used; the age, weight and condition of the patient to be treated; the route of administration; and the required regimen. Again, a physician will be able to determine the required route of administration and dosage for any particular patient. A typical daily dose is from about 0.1 to 50 mg per kg, preferably from about 0.1 mg/kg to 10 mg/kg of body weight, according to the activity of the specific inhibitor, the age, weight and conditions of the subject to be treated, the type and severity of the degeneration and the frequency and route of administration. Preferably, daily dosage levels are from 5 mg to 2 g.

The assay of the present invention is particularly advantageous because it allows flexibility in the nature and number of responsive elements being compared.

### EXAMPLES

All cell culture reagents were purchased from Life Technologies (Paisley, UK). FireLITE assay kit and white 96-well view plates were supplied by Packard Biosciences (Pangbourne, UK).

Construction of Luciferase Reporter Genes

A synthetic GR responsive promoter consisting of three copies of the consensus glucocorticoid responsive element (GRE) (AGAACAnnnTGTACC) linked to a minimal herpes simplex thymidine kinase (HSV-TK) promoter was derived from the pGRE-SEAP plasmid purchased from Clontech. This was placed upstream of the Renilla luciferase reporter gene, contained in the pRLNull vector (Promega), to create the reporter construct p3GRE-renilla. The GRE containing promoter together with the renilla luciferase gene were then subcloned into the pcDNA3 vector, deleted of the CMV promoter, in order to generate the construct p3GRE-renilla-neo.

The long terminal repeat from the mouse mammary tumour virus (MMTV) contains several GREs. This region of the viral DNA was cloned into p3GRE-renilla-neo so as to replace the synthetic GRE sequences with the MMTV promoter, thus generating pMMTV-renilla-neo

The firefly luciferase gene driven by a promoter containing five copies of the nuclear factor kB consensus sequence (GGGGACTTTCC) was derived from the pNF-kB-Luc plasmid, purchased from Stratagene, and subcloned into the pcDNA3 vector, deleted of the CMV promoter, in order to generate the construct p5NFkB-firefly-neo.

Similarly the minimal region of the Interleukin-2 promoter containing 4 AP-1 binding sites was cloned upstream of the firefly luciferase gene to generate pAP1-firefly-neo

Generation of the Firefly- and Renilla-luciferase Reporter Cell Lines

A549 cells were maintained in 5% CO₂ at 37° C. in DMEM containing 10% foetal calf serum and 2 mM L-glutamine. Cells were stably transfected with p5NFkB-firefly-neo, p3GRE-renilla-neo or pAPI-firefly-neo using electroporation procedure. Following selection in the presence of 1 mg/ml G418+Pen/strept non-essential amino acids, clones that exhibited a high reporter response, following stimulation with steroids or TNF glucocorticoids or EGF respectively, were selected and designated A549-NFkB-firefly, A549-GRE-renilla and A549-API-firefly. These cell lines were maintained in DMEM containing 10% foetal calf serum, 2 mM L-glutamine and 1 mg/ml G418.

FireLITE Assay

Cells were plated into individual wells of a white 96-well viewplate at a cell density of 4×10⁴/well in 100 μl of growth medium.

Twenty-four hours later the cells were washed once with PBS and G418 free quiescence medium (DMEM containing penicillin/streptomycin and non-essential amino acids, 2 mM L-glutamine) was added in the presence or absence of compounds. One hour later stimulation was performed by adding TNF in a total volume of 100 μl/well. Following 16 hours incubation at 37° C., the cells were washed once with PBS and 100 μl/well of PBS containing 1 mM Ca⁺⁺ and Mg⁺⁺ ions was added. Subsequently 100 μl of reconstituted LucLITE substrate was added to each sample, the plates incubated at room temperature for 10 minutes, and firefly luciferase luminescence measured on the Topcount at 22° C. in luminescence mode. To measure Renilla luciferase activity, the plate was removed from the Topcount and 50 μl of reconstituted RenLITE substrate added to each well. Following a further 30 min incubation at room temperature, Renilla luminescence was measured on the Topcount as before.

Individual Cell Clone Characterisation

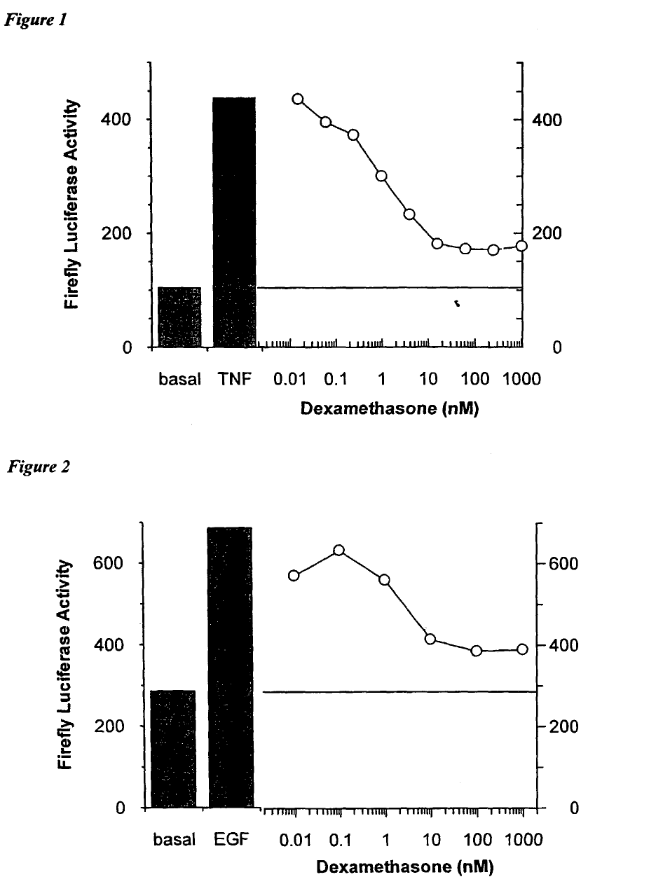

The individual cell clones were first characterised for responsiveness in isolation. Stimulation of the A549-NFkB-firefly cells with 0.5 ng/ml TNFα resulted in a 4.2 fold increase in firefly luciferase activity (FIG. 1). Treatment of the cells with the standard glucocorticoid receptor agonist, dexamethasone, for 1 hour before and during stimulation with TNFα resulted in a dose-dependent inhibition of NFkB driven firefly luciferase activity (FIG. 1). This demonstrates that this cell line has utility in monitoring the repression of NFkB signalling by glucocorticoid receptor ligands.

Similarly, expression of firefly luciferase in the A549-AP1-firefly cells was increased 2.4 fold following stimulation of the cells with 10 ng/ml EGF, and this increase in activity was inhibited dose-dependently by dexamethasone (FIG. 2). This cell line can therefore be used to monitor the repression of AP-1 signalling by glucocorticoid receptor ligands.

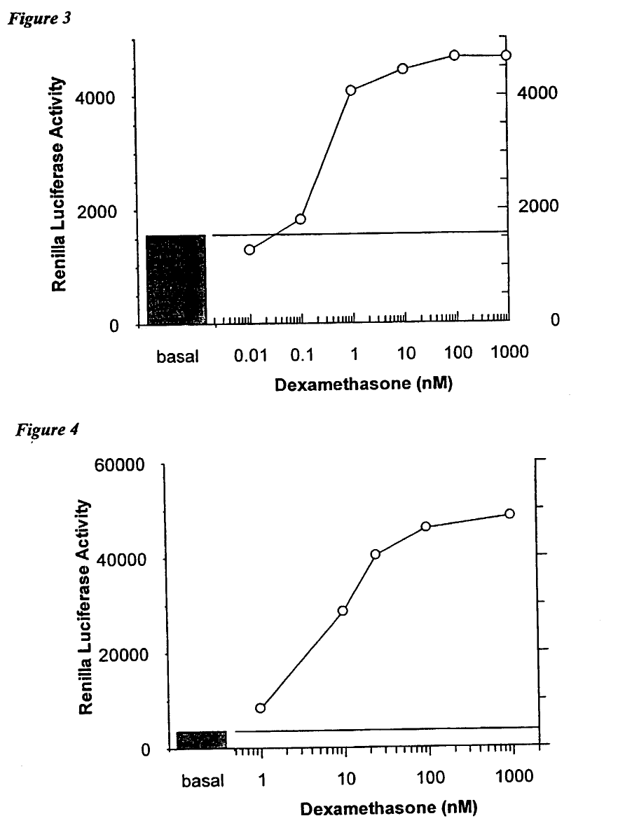

Treatment of the A549-GRE-Renilla cells with dexamethasone resulted in a dose-dependent increase in renilla luciferase activity, with a maximal stimulation above basal of 3 fold (FIG. 3). This cell line will provide a means of measuring direct effects of glucocorticoid receptor agonists on GRE-mediated gene transcription.

The MMTV-renilla construct was introduced into COS-1 cells along with a polynucleotide construct encoding the human glucocorticoid receptor. When these cells were stimulated with dexamethasone, a 13 fold increase in luciferase activity was observed (FIG. 4). This demonstrates that the MMTV-renilla construct is an alternative way of monitoring GRE-mediated gene transcription, and indicates that other cell lines, including those lacking endogenous GR can be utilised.

Dual Cell Assay

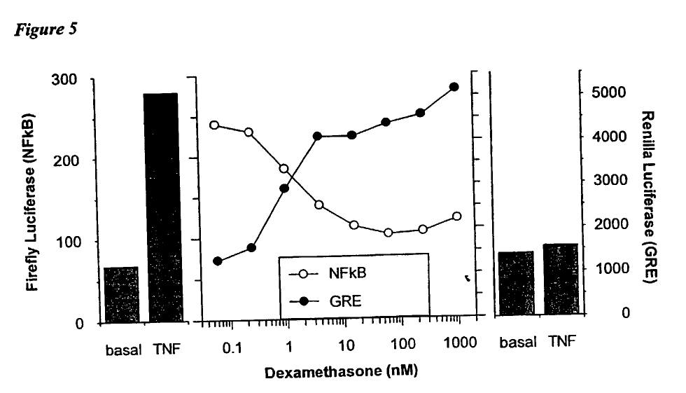

The A549-NFkB-Firefly and A549-GRE-Renilla cell lines were mixed in a ratio 1:1 and assayed in dual assay format. Under these conditions, each cell line behaved exactly as they had in isolation (FIG. 5), demonstrating the usefulness and flexibility of this format.

1. A method of assessing the glucocorticoid receptor signalling pathway modulated by a test compound comprising;
(a) providing a cell having a first polynucleotide construct comprising a first reporter gene under the control of a first promoter indicative of a first glucocorticoid receptor signalling pathway;
(b) providing a second cell of the same lineage as the cell (a), and comprising a second polynucleotide construct having a second reporter gene under the control of a second promoter indicative of a second glucocorticoid receptor signalling pathway;
(c) incubating said first and second cells with a test compound;
(d) monitoring for expression of the first and second reporter genes.

2. A method according to claim 1 wherein the first and/or second promoters comprise a responsive element indicative of the signalling pathway under investigation.

3. A method according to claim 2 wherein the first and/or second promoter comprises the glucocorticoid responsive element, or a transcription factor recognition site.

4. A method according to claim 3 wherein the transcription factor recognition site is selected from an AP-1, NF-6B, C/EBP-1, STAT-5, Oct-1 or Oct-2 recognition site.

5. A method according to claim 2, 3 or 4 wherein the first and/or second promoter comprises a non-naturally occurring promoter comprising a minimal promoter together with the responsive element.

6. A method according to any one of the proceeding claims wherein the first and second reporter genes are detectable by fluorogenic, luminometric, or colourometric means.

7. A method according to claim 6 wherein both reporter genes are detectable by luminometric means.

8. A method according to claim 7 wherein the first reporter gene comprises firefly-luciferase and the second reporter gene comprises renilla-luciferase.

9. A method according to any one of the proceeding claim wherein both the first and second polynucleotide constructs are provided in the same cell.

10. A method according to claim 2 wherein the first signalling pathway comprises indirect inhibition of a transcription factor and the second signalling pathway comprises direct DNA binding.

11. The method according to any one of the preceding claims which further comprises providing a third cell of the same lineage as the cell (a) and (b) and comprising a third polynucleotide construct having a third reporter gene under the control of a third promoter indicative of a third glucorticoid receptor signalling pathway, incubating said third cell with a test compound and monitoring for expression of the third reporter gene.

12. A compound identified by the method of claim 10, wherein said compound inhibits the first signalling pathway, and does not activate the second signalling pathway for use in the treatment of rheumatoid arthritis, osteoarthritis, inflammatory pain, emesis, inflammatory bowel disease, asthma, atopic dematitis and psoriasis.


SEQUENCE LISTING (2 sequences)

SEQ ID NO: 1 - DNA, 15 bp, Artificial Sequence
  Synthetic GRE construct
agaacannntgtacc

SEQ ID NO: 2 - DNA, 11 bp, Firefly
ggggactttcc